(19) United States
(12) Patent Application Publication
(10) Pub. No.: US 2023/0145497 A1
(43) Pub. Date: May 11, 2023

(54) STRAIGHT-TUBE UV LIGHT

(71) Applicant: Shenzhen Guanke Technologies Co., Ltd, Shenzhen (CN)

(72) Inventors: Qing LAN, Shenzhen (CN); Shoubao Chen, Shenzhen (CN); Bo Lei, Shenzhen (CN); Jinliang Lei, Shenzhen (CN); Tianlong Dai, Shenzhen (CN); Ligen Liu, Shenzhen (CN); Minggui Wang, Shenzhen (CN)

(73) Assignee: Shenzhen Guanke Technologies Co., Ltd, Shenzhen (CN)

(21) Appl. No.: 17/571,755

(22) Filed: Jan. 10, 2022

(30) Foreign Application Priority Data
Nov. 5, 2021 (CN) 2021227192725

(51) Int. Cl. A61L 2/10; F21V 15/015; F21V 29/83; F21V 23/00; F21V 7/04; A61L 2/24; A61L 9/20

(52) U.S. Cl. CPC A61L 2/10; F21V 15/015; F21V 29/83; F21V 23/003; F21V 7/04; A61L 2/24; A61L 9/20; F21Y 2103/00

(57) ABSTRACT

A straight-tube UV light. The straight-tube UV light includes a light body, end covers, and functional subassemblies. The light body is of a hollow tubular structure provided in a straight line; the end cover includes a first end cover and second end cover provided on a left and right of the light body, the first end cover, the second end cover and the light body enclose to form a mounting cavity. The functional subassemblies include at least the first light source subassembly and the control power supply, provided inside the mounting cavity, the control power supply controls the first light source subassembly to emit UV rays for sterilization and disinfection. This can simplify the protective structure of the light, reduce the volume of the light, and improve a sealing property of the light.

### TECHNICAL FIELD

The present invention relates to the technical field of lights, particularly to a UV light.

### BACKGROUND

Strong-UV lights refer to medium-power or large-power UV lights whose power is above 20 W. Their strong UV rays can achieve good sterilization and disinfection effects, can also bring bigger damage to human bodies.

In existing technologies, to prevent strong UV rays of lights from irradiating human bodies, UV rays are usually designed into a large-dimension cabinet structure by adding sealing or shielding parts around the UV light source, fan, control power supply, optical subassembly or other functional components and parts. Such parts are usually set in different cavities, leading to repeated multi-layer protection. The design scheme of repeated protection will lead to a complex structure and a bigger volume of the light. Also, the main structure of existing UV lights is not suitable for deriving different types of UV lights, and it is hard to realize unification of key parts of different types of UV lights.

The foregoing content is only used for assisting in understanding the technical scheme of the present invention, but does not mean the acknowledgement that the above content is the prior art.

### SUMMARY

To solve the above problem, the present invention provides a straight-tube UV light that can not only simplify the protective structure of the light, reduce the volume of the light and improve the sealing property of the light, but can also derive other types of UV lights to realize standardization and unification of key components and parts of different types of UV lights.

The straight-tube UV light provided by the present invention comprises:

a light body of a hollow tubular structure provided in a straight line, defined as that the length of the light body extends from the left to the right, height extends from the up to the bottom and width extends from the front to the back;

end covers, including the first end cover and the second end cover provided on the left end and the right end of the light body, the first end cover, the second end cover and the light body enclosing to form a mounting cavity, the first end cover and the second end cover respectively provided with the first air hole and the second air hole connecting to the mounting cavity;

a functional subassembly comprising at least the first light source subassembly and the control power supply, the functional subassembly is wholly provided in the mounting cavity, the control power connecting to the first light source subassembly, the control power supply used for controlling UV rays irradiated by the first light source subassembly for sterilization and disinfection.

Other characteristics and corresponding beneficial effects of the present invention are elaborated in the latter part of the description.

The solving ideas of technical problems of the present invention and relevant product design solutions are as shown below:

The light body of the UV light is designed into a straight tubular structure. The light body is of a hollow tubular structure in a straight line shape. Each of two ends of the light body is provided with an end cover, the light body and two ends cover enclose each to form a mounting cavity that serves as the main structure of the light. Functional components and parts (i.e. functional subassemblies) like the UV light source, fan, control power supply or optical modules are provided in the mounting cavity. All the functional components and parts are protected, stored and mounted in a unified way via the main structure to significantly reduce the volume of the light, simplify the protective structure of the light and improve the sealing property of the product. Functional components and parts are mounted in the mounting cavity, by which components and parts are less likely to be exposed, and then the appearance of the light is more concise and beautiful.

Additionally, under such a straight-tube UV light design scheme, functional components and parts are harder to mount due to the thin and long space of the tubular light body, but the light can derivate different lengths and power sizes of UV lights as well as different types of UV lights (for example, ventilation-type UV lights can be derived into upper-layer flat irradiation UV light or direct irradiation UV light.

Definition and characteristics of different types of UV lights:

(1) Direct irradiation UV light: The light directly exposes UV rays to an open environment for sterilization and disinfection of object surfaces and air, achieving a high sterilization and disinfection effect; however, people should not stay in the sterilization environment when the light is working, so the safety performance is low.

(2) Ventilation-type UV light: Such lights inhale air into the enclosed light cavity, after UV sterilization and disinfection in the light cavity, sterilized air is exhausted out of the light to finish air sterilization and disinfection and ventilation. They are mainly used for air disinfection. People do not need to leave the sterilization place when such lights are working, so people can be protected from the damage of UV rays, man-machine symbiosis can be realized, and the safety performance is high; however, the sterilization efficiency is low, noises are likely to occur during ventilation, and products are usually designed into large-size cabinets, so they have a large volume and a heavy weight.

(3) Upper-layer flat irradiation UV light: By mounting the UV light to an upper space that is 2.1 m above the ground or floor, the UV light can irradiate UV rays horizontally to sterilize and disinfect the upper space. During disinfection with the light, people can act in the lower space below 2.1 m, and do need to leave the sterilization place, enabling a high safety level. However, the product is usually designed into a large-size cabinet structure featured with a big volume and heavy weight.

During implementation of the product, the light body can be made of aluminum alloy profile that is easy to cut into different lengths. To facilitate installation of corresponding parts into a thin and long space, the inner surface of the light body is provided with a chute structure. The UV light source, control power supply or optical subassembly can be mounted onto the support in advance, and then the support is pushed to the corresponding position along the chute to realize assembly of corresponding parts. All the parts can be mounted with different supports, and can share one support. When the UV light is provided with a fan, the fan can be fixed on to the end cover in advance, and then the end cover can be fixed onto the end of the light to realize mounting of the fan. For example, the end cover is provided with a mounting column, the fan is fixed onto the mounting column, which can reduce noises caused by vibration of the fan arising from installation of the fan onto the support.

As shown in FIG. 1, when the UV light is an upper-layer flat irradiation UV light, the front side of the light body is provided with a light outlet. The mounting cavity opposite to the light outlet is provided with a grating subassembly that distributes light to make the UV rays emit in the horizontal direction. Both the front side and the rear side of the light body can be provided with a light outlet. The front light outlet and the rear light outlet are provided with a grating subassembly. The light is designed into an upper-layer flat irradiation UV light that can emit light in two opposite directions.

As shown in FIG. 14, when the UV light is a ventilation-type UV light, there is no additional opening on the upper surface, lower surface, front surface or rear surface of the light body, and either of the first air hole and or the second air hole serves as the air inlet, and the other serves as the air outlet. And, the corresponding position of the first air hole and the second air hole is provided with an anti-exposure structure (e.g. the filter structure 24 and baffle 3 in the embodiment), avoiding leakage of UV rays in the mounting cavity and improving the service safety of the light.

As shown in FIG. 24, the UV light enables properties of both the ventilation-type UV sterilization and the direct irradiation UV sterilization. This direct irradiation scheme may be direct UV sterilization or direct lighting. The lower side of the light body is provided with the second opening, and the light body is provided with a dismountable second cover plate for sealing the second opening. The second cover plate is elongated. After opening the second cover plate, components and parts inside the mounting cavity can be easily mounted or dismounted, including the first light source subassembly and the control power supply. This is another scheme for mounting functional components and parts inside a small thin and long space. Functional components and parts can be mounted onto the upper surface of the second cover plate or the inner surface of the light body. The lower surface of the second cover plate is provided with the second light source module. The first light source module applies UV light source. The second light source module applies UV light source or white light source to emit corresponding UV rays or visible light.

Among the above-mentioned several types of UV lights, the first end cover, the second end cover, fan, UV light source, control power supply, support and other key parts can be designed into common standard parts. And, the light body can be made of aluminum profile. Except the light outlet on the light body or the second outlet that needs post-processing, the dimensions and structure of the chute and other mounting components inside the light may be unified to realize unified standards of the light mold. Such a straight-tube UV light straight-tube UV light design scheme is very suitable for standardizing parts of different types of lights, by which the cost can be significantly reduced via batch production, strong-UV lights may be widely applied by common civil sterilization, solving some existing industry problems like big volume and expensive price of strong-UV lights. Such a design scheme has a new application prospect.

### BRIEF DESCRIPTION OF THE DRAWINGS

FIG. 1 is a schematic diagram showing the structure of an embodiment of the straight-tube UV light of the present invention based on the upper-layer flat irradiation UV light design scheme;

FIG. 2 is a schematic diagram showing the breakdown of a local part of the straight-tube UV light in FIG. 1;

FIG. 3 is a schematic diagram showing the breakdown of another local part of the straight-tube UV light in FIG. 1;

FIG. 4 is the a schematic diagram showing the structure of the grating subassembly in FIG. 2;

FIG. 5 is a schematic diagram showing the structure of the cross section of A-A in FIG. 4;

FIG. 6 is a schematic diagram showing the structure of the cross section of the baffle in FIG. 5;

FIG. 7 is a schematic diagram showing the structure of baffle in FIG. 4;

FIG. 8 is a schematic diagram showing the structure of the inside of the straight-tube UV light in FIG. 1;

FIG. 9 is a schematic diagram showing the structure of the inside of the straight-tube UV light in FIG. 1;

FIG. 10 is another schematic diagram showing the structure of the inside of the straight-tube UV light in FIG. 1;

FIG. 11 is a schematic diagram showing the structure of the cross section of the light body in FIG. 2;

FIG. 12 is a schematic diagram showing the explosive view of the sensing module in FIG. 3;

FIG. 13 is a schematic diagram showing the explosive view of the sensing module in FIG. 3 from another perspective;

FIG. 14 is a schematic diagram showing the breakdown structure of an embodiment of the straight-tube UV light of the present invention based on the ventilation-type UV light design scheme;

FIG. 15 is a schematic diagram showing the structure of the cross section of the straight-tube UV light in FIG. 14;

FIG. 16 is a schematic diagram showing the structure of the cross section of the straight-tube UV light in FIG. 15 from another perspective;

FIG. 17 is a schematic diagram showing the structure of assembling parts in FIG. 16;

FIG. 18 is a schematic diagram showing the structure of light cap in FIG. 15;

FIG. 19 is a schematic diagram showing the structure of the baffle of another embodiment in FIG. 15;

FIG. 20 is a schematic diagram showing the structure of the inside of the straight-tube UV light in FIG. 15;

FIG. 21 is a schematic diagram showing the structure of the opening face of the straight-tube UV light in FIG. 15;

FIG. 22 is a schematic diagram showing the amplifying structure of position B in FIG. 21;

FIG. 23 is a schematic diagram showing the structure of the light body of another embodiment in FIG. 14;

FIG. 24 is a schematic diagram showing the structure of an embodiment of the straight-tube UV light of the present invention based on the ventilation-type and direct irradiation UV light design scheme;

FIG. 25 is a schematic diagram showing the structure of another service state of the straight-tube UV light in FIG. 24;

FIG. 26 is a schematic diagram showing the structure of the cross section of the straight-tube UV light in FIG. 24;

FIG. 27 is a local schematic diagram of the straight-tube UV light in FIG. 24;

FIG. 28 is a schematic diagram showing the structure of the light body in FIG. 24.

The shape, dimension, proportion or position relationship of parts of the product in drawings may be real data of embodiments and they are under protection of the present invention.

### DETAILED DESCRIPTION

To make the objective, technical solutions and advantages of the present invention clearer and be understood better, further detailed descriptions of embodiments of the present invention are made in combination with drawings. Understandably, the specific embodiments described are just used to explain but not limit the present invention.

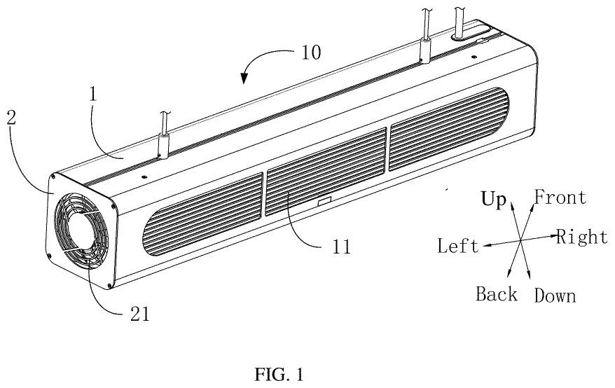

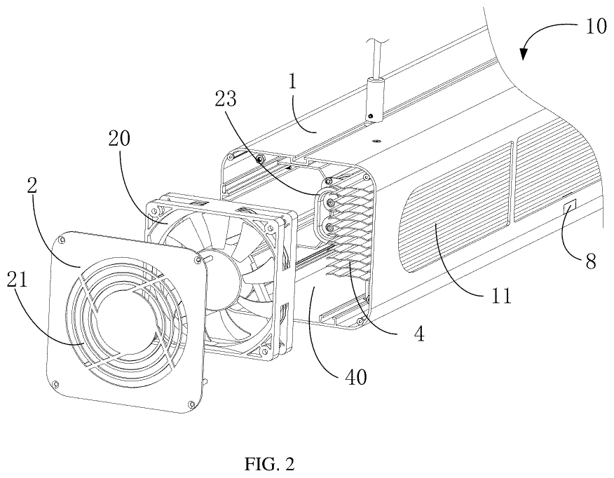

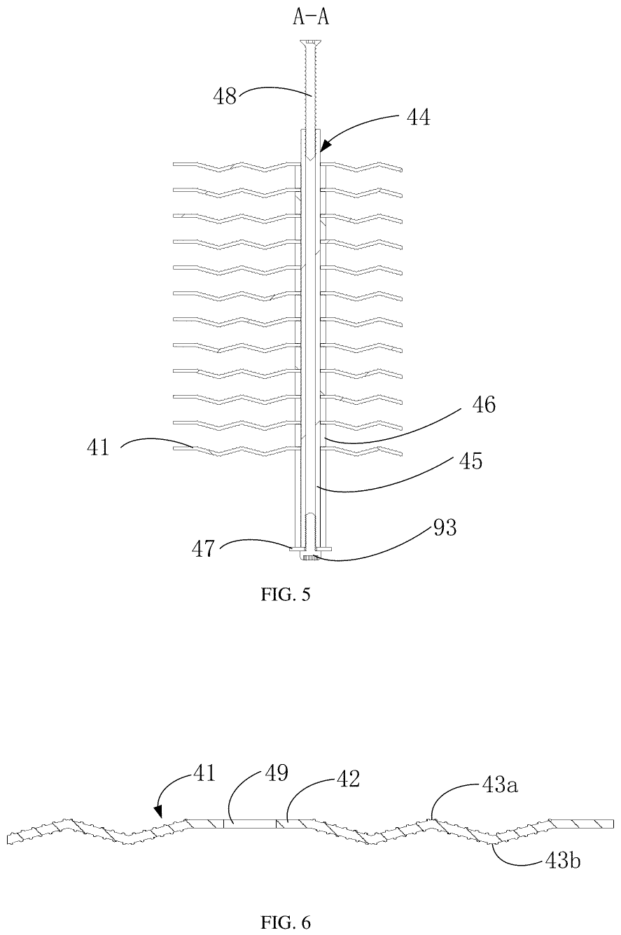

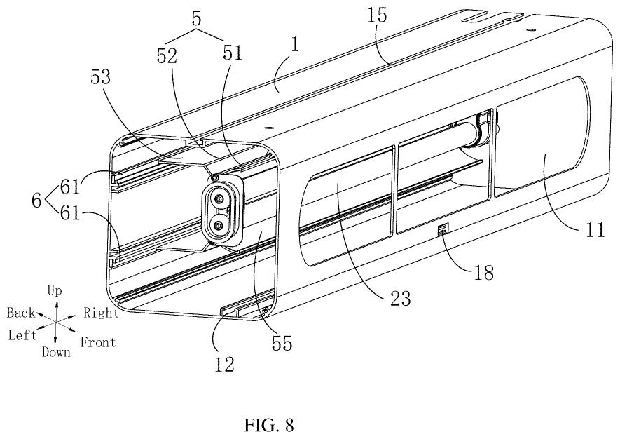

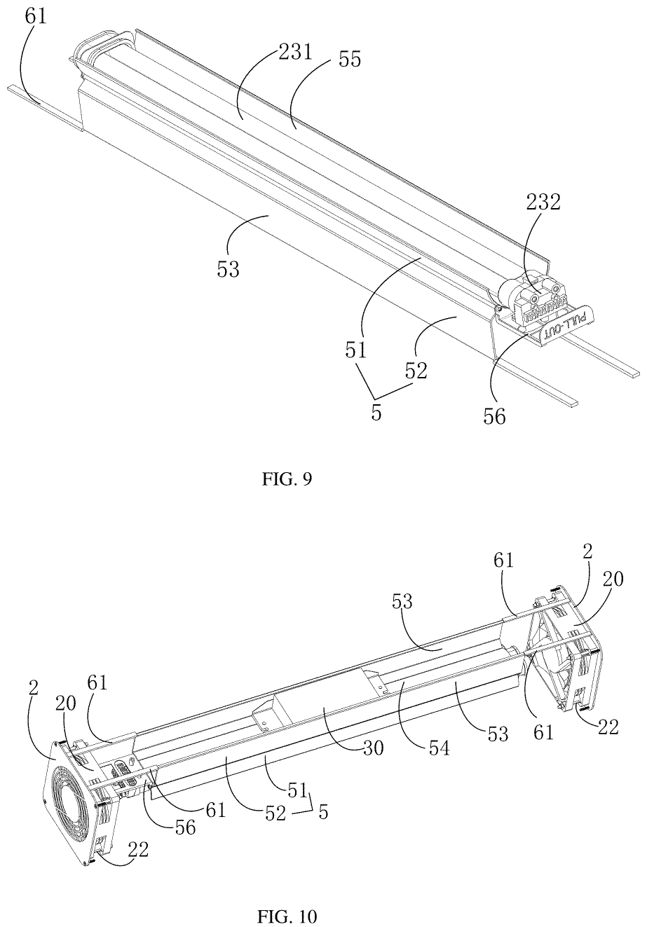

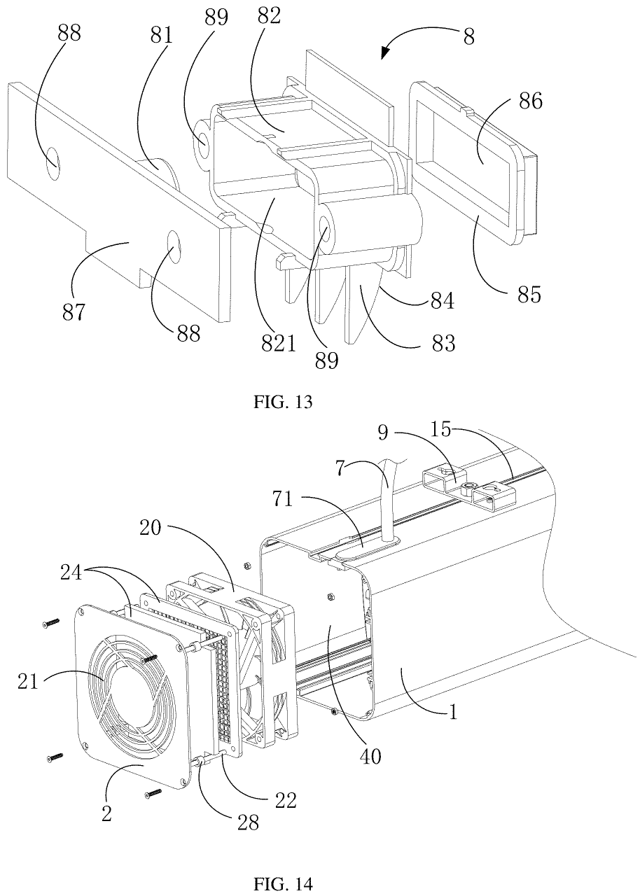

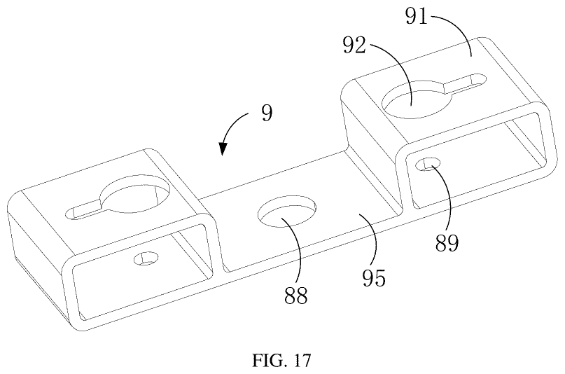

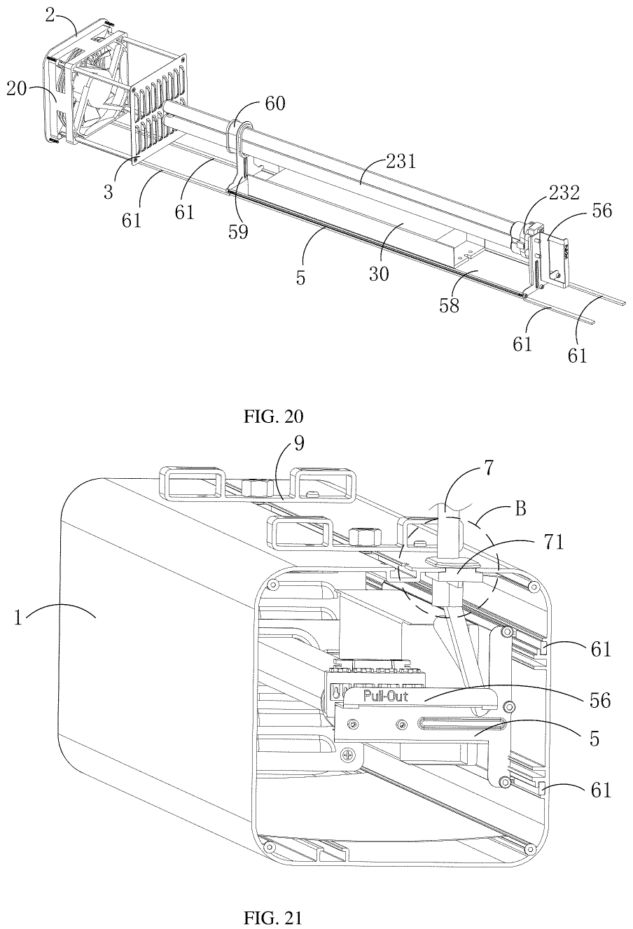

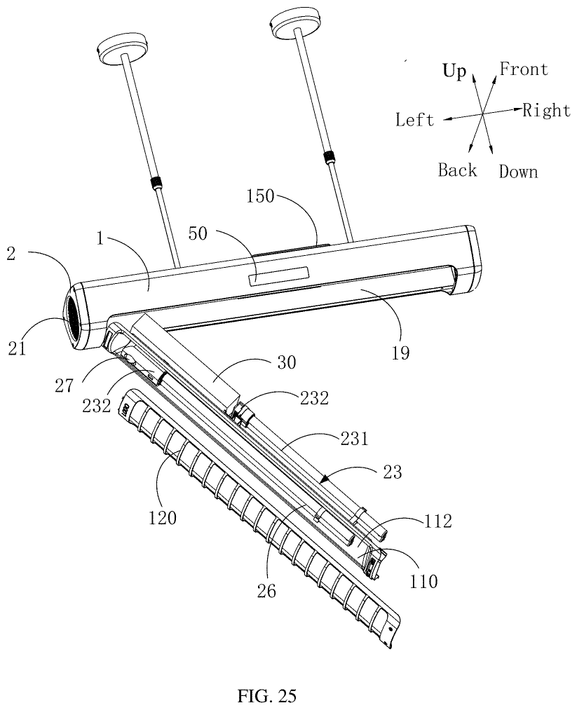

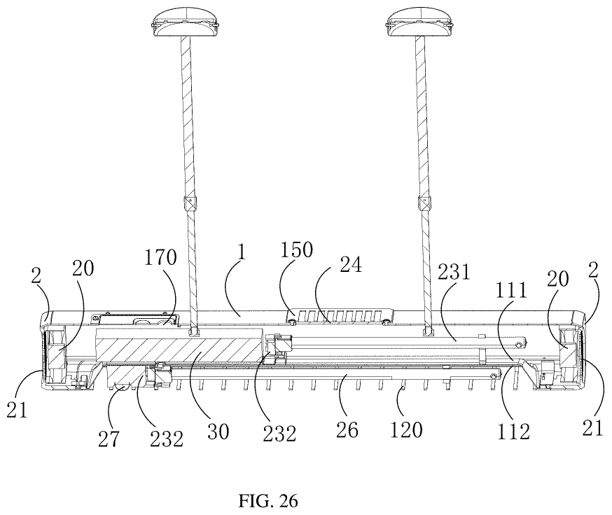

As shown in FIG. 1-FIG. 28, the present invention provides a straight-tube UV light 10 that mainly comprises the light body 1, end covers 2 and functional subassemblies; wherein, the light body 1 is a hollow tubular structure in a straight line shape, and it is defined that the length extension direction of the light body 1 is the left-right direction, the height extension direction is the up-down direction, and the width extension direction is the front-rear direction; end cover 2 comprises the first end cover 2 and the second end cover 2 that cover the left opening and the right opening of the light body 1 respectively, forming the mounting cavity 40 with the light body 1, the first end cover 2 and the second end cover 2 are respectively provided with the first air hole 21 and the second air hole 21 that connect the mounting cavity 40 and the outside; all the functional subassemblies are provided inside the mounting cavity 40, functional subassemblies at least include the first light source subassembly 23 and control subassemblies, and control subassemblies connect to the first light source subassembly 23 to control UV rays emitted by the first light source subassembly 23 for sterilization and disinfection.

The light body 1 of the UV light 10 is designed into a straight tubular structure. The light body 1 is of a hollow tubular structure in a straight line shape. Each of two ends of the light body 1 is provided with an end cover 2, the light body 1 and two ends cover 2 enclose each to form a mounting cavity 40 that serves as the main structure of the light. Functional components and parts (i.e. functional subassemblies) like the UV light source, fan 20, control power supply 30 or optical modules are provided in the mounting cavity 40. All the functional components and parts are protected, stored and mounted in a unified way via the main structure to significantly reduce the volume of the light, simplify the protective structure of the light and improve the sealing property of the product. Functional components and parts are mounted in the mounting cavity 40, by which components and parts are less likely to be exposed, and then the appearance of the light is more concise and beautiful.

The upper-layer flat irradiation straight-tube UV light 10 is as shown in FIG. 1-FIG. 13.

As shown in FIG. 1-FIG. 3, in one embodiment, functional subassemblies also include the grating subassembly 4 in the mounting cavity 40, the front side of the light body 1 is provided with the first opening 11, the first opening 11 is opposite to the first light source subassembly 23, the grating subassembly 4 is provided between the first light source subassembly 23 and the first opening 11; wherein, grating subassembly 4 comprises several parallel baffles 41 provided at intervals and horizontally, two adjacent baffles 41 form a light emitting slot, the first light source subassembly 23 emits UV rays along the light emitting slot to the outside of the light body 1 for sterilization and disinfection. The surface of baffles 41 is provided with a light absorption layer that can be black UV ray absorption materials with oxidized anode or coated UV ray absorption materials; before entering the light emitting slot, UV rays are scattered, UV rays with a big included angle with the light emitting direction will be intercepted by baffles 41 or absorbed by the surface of baffles 41, so that UV rays emitted by the light will be emitted roughly in the horizontal direction.

Further, the side of the end cover 2 on one side or both sides nearby the mounting cavity 40 is provided with a support pillar 22; functional subassemblies include the fan 20 that is fixed onto the end cover 2 via the support pillar 22, the fan 20 is opposite to the air hole 21, that is, only one end cover 2 can be provided with one fan 20, or both end covers 2 can be provided with a fan 20 respectively; when two fans 20 are adopted, they can inhale air into the light at the same time, and then air is exhausted out of the light via the grating subassembly 4; or air can be exhausted via end covers 2 and inhaled via the light outlet of the grating subassembly 4 at the same time; in such a way, air convection can be accelerated, and the sterilization and disinfection efficiency is improved.

In another embodiment, the inside of the light may be not provided with a fan 20, end covers 2 on both ends may be not provided with an air hole 21, surrounding air is sterilized and disinfected only with UV rays emitted via baffles 41, because the effective sterilization distance of UV rays emitted horizontally by the strong UV light source whose power is above 20 W can be above 5 m, and the sterilization effect is very good.

In one embodiment, both the front side and the rear side of the light body 1 are provided with a light outlet, two light outlets are provided with two corresponding grating subassemblies 4, UV rays emitted by the first light source subassembly 23 go out of the light body 1 respectively via two grating subassemblies 4, improving the UV sterilization scope and efficiency.

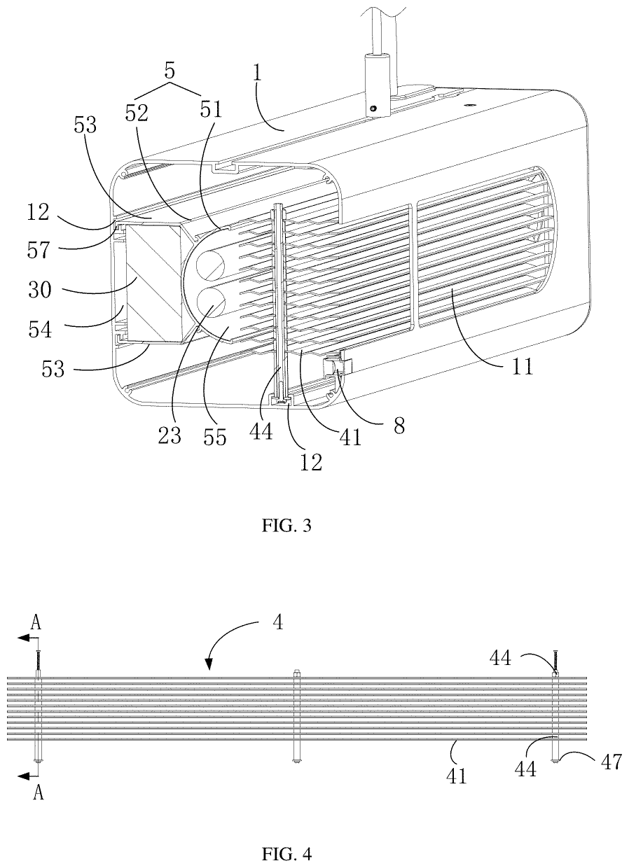

From FIG. 3-FIG. 5, in one embodiment, the grating subassembly 4 at least includes one connecting structure 44, each connecting structure 44 includes one central column 45 and multiple connecting columns 46, several parallel baffles 41 and several connecting columns 46 enclose the central column 45 at intervals, enabling multiple baffles 41 to be provided at the light outlet evenly and more stably.

Wherein, the bottom of the central column 45 is provided with a sliding part 57. Correspondingly, the inner side of the light body 1 is provided with a chute structure 12. The sliding part 57 and the chute structure 12 collaborate with each other, enabling the grating subassembly 4 to move to the preset assembly position along the chute structure 12. The grating subassembly 4 is fixed onto the light body 1 in a dismountable way via the locating device 6.

Specifically, the central column 45 is a hollow structure. One gasket 47 is fixed to the bottom of the central column 45 via the bolt 93, and the gasket 47 serves as one sliding part 57 to make the grating subassembly 4 slide inside the light body 1; the locating device 6 may be several trim strips 61 provided inside the chute, after the grating subassemblies 4 slide to the preset position, one end of the trim strip 61 holds onto the sliding part 57, the other end holds onto the end cover 2 to fix the connecting structure 44; or bolts or other fastening parts are added between the light body 1 and the connecting structure 44 to fix grating subassembly 4.

In another embodiment, the bottom of the central column 45 may be provided with one stud bolt 93 whose head is provided inside the chute structure 12 and serves as the sliding part 57 to make the grating subassembly 4 slide inside the light body 1.

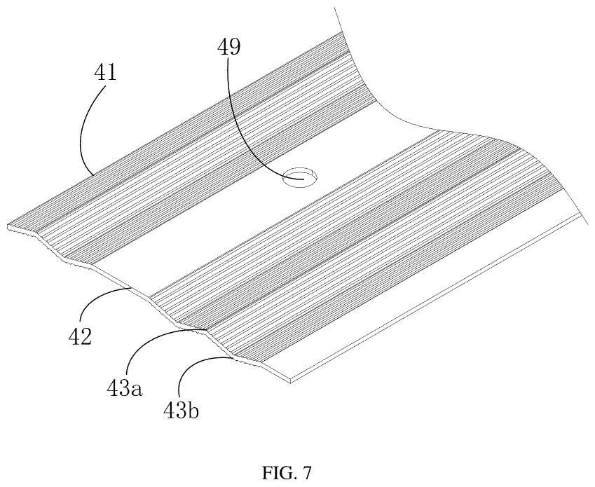

As shown in FIG. 6 and FIG. 7, in one embodiment, the body 42 of baffles 41 is provided in a curve shape to form multiple convex 43a on the surface 111 of baffles 41 and multiple convex 43b on the lower surface 112 of baffles 41, and at least partial upper convex 43a and lower convex 43b are connected in sequence to form a consecutive waveform structure; baffles 41 may include a horizontal baffle and two consecutive waveform structures, the horizontal baffle as the middle part of baffles 41 is provided horizontally, and two consecutive waveform structures are connected by horizontal plate bodies to form baffles 41; the middle of the horizontal plate bodies is provided with a mounting hole 49 that is used for fixing baffles 41. Compared with flat baffles 41, baffles 41 in this embodiment can intercept a large angle of UV rays segments by segments, make other UV rays in non-horizontal directions be gradually attenuated and filtered, so that the luminous angle of grating subassembly 4 can be reduced without increasing the width of baffles 41 or narrowing the spacing of baffles 41; second, by adopting waveform baffles 41, it is easier for the light to realize the safety level of the exempt group in the IEC 62471 Light and Light System Photobiological Safety and achieve good application values.

Specifically, baffles 41 are about 0.8 mm thick and 60 mm wide, the spacing of baffles 41 is 6.8 mm, the height difference between the top of the upper convex 43a and the bottom of the lower convex 43b is greater than 1.5 mm, which enables other non-horizontal UV rays to be attenuated and filtered better. Of course, there will be a certain error in actual manufacture, which should be regarded as being within the scope of the present invention.

As shown in FIG. 3 and FIG. 8-FIG. 10, in one embodiment, functional subassemblies also include the support 5 provided inside the mounting cavity 40, and the first light source subassembly 23 and the control power supply 30 are fixed onto the support 5 respectively.

Specifically, support 5 is composed of the reflective part 51 and the connecting part 52, the reflective part 51 is an arc structure, the concave surface of the reflective part 51 faces the first opening 11, the first light source subassembly 23 provided inside the convex surface of the reflective part 51 reflects UV rays other than those from the first opening 11 to improve the quantity of UV rays form the first opening 11.

The inner surface of the light body 1 is provided with a chute structure 12, the bottom of the connecting part 52 is provided with a sliding part 57, the sliding part 57 and the chute structure 12 collaborate with each other to make the support 5 move to the preset assembly position along the chute structure 12 and make the support 5 fix into the light body 1 in a dismountable way via the locating device 6, facilitating dismounting of support 5 and further inspection, maintenance or repair of functional subassemblies. wherein, the locating device 6 may be several trim strips 61 provided inside the chute, after the support 5 slides to the preset position, one end of the trim strip 61 holds onto the sliding part 57, the other end holds onto the end cover 2 to fix the support 5; or bolts or other fastening parts are added between the light body 1 and the support 5 to fix support 5.

If it is defined that the concave surface of the reflective part 51 is the front, the opposite surface of the concave surface is the back, the connecting part 52 comprises two supporting arms 53 provided at intervals, two supporting arms 53 are provided on the back of the reflective part 51 to form the first mounting slot 54, and the control power supply 30 is fixed inside the first mounting slot 54; sliding part 57 is provided on one end of each supporting arm 53 far from the reflective part 51.

Further, the reflective plate 55 is provided against the concave surface of the reflective part 51 to improve the reflection efficiency of UV rays. Wherein, the reflective plate 55 can be mirror-surface metal sheet that can reflect UV rays better.

Further, straight-tube UV light 10 also comprises a handle 56 provided on one end of support 5, the handle 56 drives the support 5 to push or pull the light body 1 along the chute structure 12, facilitating the user to operator.

Further, the first light source subassembly 23 comprises the first light source 231 and the mounting base 232, the mounting base 232 is fixed onto the handle 56, the mounting base 232 is used to fix the first light source 231 and supply power for the first light source 231, the first light source 231 and the reflective part 51 are provided at intervals, that is, there is a certain clearance between the first light source 231 and the reflective part 51, enabling UV rays emitted by the first light source 231 onto the reflective part 51 to be reflected better.

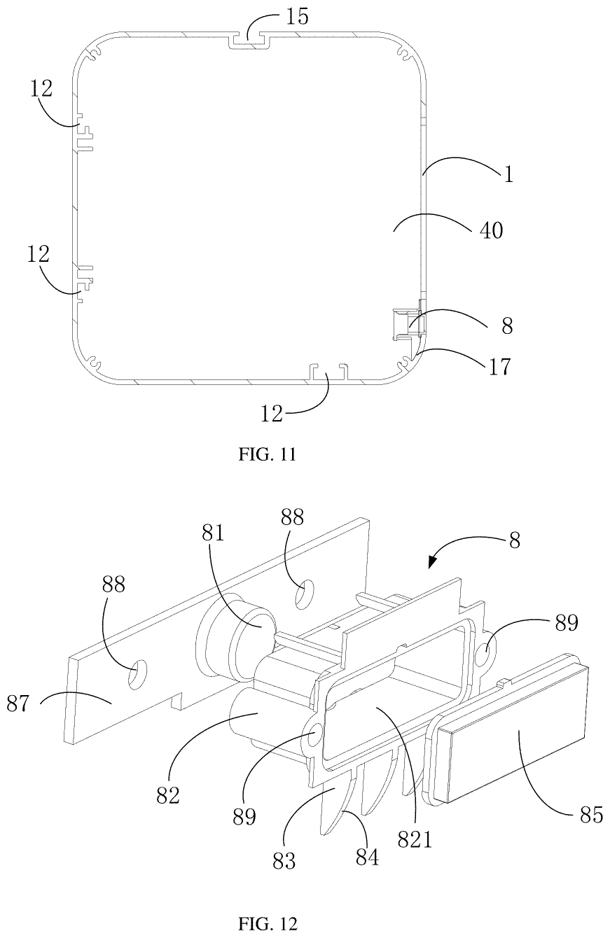

As shown in FIG. 11-FIG. 13, in one embodiment, the straight-tube UV light 10 also comprises a sensing module 8 that electrically connects to the control power supply 30 and is provided under the front surface of the light body 1; wherein, the sensing module 8 is an infrared sensing module that only receives horizontal sensing signals, when the human body reaches the sensing height of the sensing module 8, the sensing module 8 will automatically turn off the first light source 231. The sensing module 8 is provided under the front surface of the light body 1, that is, it is provided below the UV rays emitted, to monitor human body before the human body enters the UV ray irradiation scope, so that it can better prevent human body being hurt by UV rays in the irradiation scope.

Wherein, the sensing module 8 comprises the sensing element 81, fixing base 82, lens 85 and circuit board 87, the sensing element 81 is provided on the circuit board 87, fixing base 82 is provided on the first pass-through slot 821, circuit board 87 is fixed onto the fixing base 82, sensing element 81 is provided inside the first pass-through slot 821, lens 85 covers the other end of first pass-through slot 821 in a fixed way to cover the light outlet of the first pass-through slot 821; lens 85 is a semitransparent or transparent material, its inner surface is provided with many kinds of optical convex patterns that enable the sensing probe to receive horizontal sensing signals and vertical sensing signals to be significantly weakened.

The inner surface of the light body 1 is provided with the first arc surface 17, the fixing base 82 comprises one or several parallel convex blocks 83 where there is the second arc surface 84, the second arc surface 84 is against the first arc surface 17, the lower part of the front surface of the light body 1 is provided with the second pass-through slot 18, the lens 85 is inserted into the second pass-through slot 18, by which the sensing module 8 is fixed onto the light body 1. This fixing mode is simpler and more efficient, and no other fixing structures are needed to fix the sensing module 8.

Further, the lens 85 is square, the side of the lens 85 opposite to the sensing element 81 is provided with a square slot 86 whose bottom wall surface is provided with circles of convex patterns which further weaken vertical sensing signals.

The ventilation-type straight-tube UV light 10 is as shown in FIG. 14-FIG. 23.

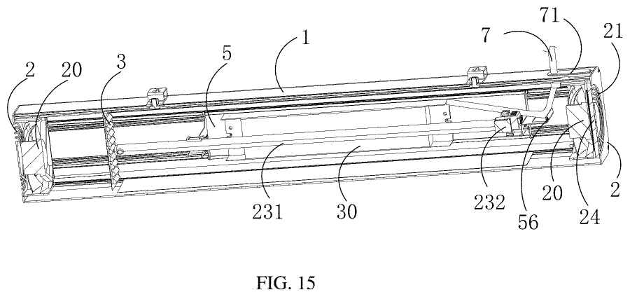

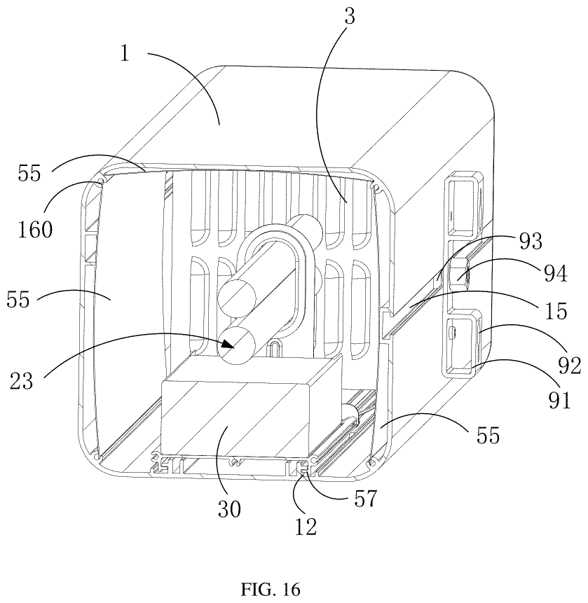

As shown in FIG. 14 and FIG. 15, in one embodiment, functional subassemblies include fan 20 provided inside the light body 1 for conveying air; except two open ends, other parts of the light body 1 is enclosed, one of the first air hole 21 and the second air hole 21 is used for flow-in of air, and the other is used for flow-out of air, forming a horizontal and straight air duct that serves as the channel for flow-in and flow-out of air during sterilization of the light. Compared with existing design, such a flat and straight air duct is relatively short and is just a little longer than the UV light source, which can shorten the path for flow-in and flow-out of air and effectively reduce the volume of the light; in addition, since the light body 1 is a straight barrel, the flow-in and flow-out resistance is small, and the sealing property is good, preventing air leakage when fan 20 runs and improving the ventilation efficiency of the UV light 10. In this embodiment, the power of the UV light source is 100 W, but the light dimension is only 140×140×900 mm.

Wherein, the first air hole 21 or the second air hole 21 is the combination of several thin and long holes whose width is smaller than 6.5 mm, preventing human body contacting internal electrified body. The light body 1 can be made of aluminum profile via the extrusion process.

In one embodiment, the quantity of fan 20 is one or two; when there is one fan 20, the side of the first end cover 2 nearby the mounting cavity 40 is provided with a support pillar 22, the fan 20 is fixed onto the first end cover 2 via the support pillar 22; when there are two fans 20, the side of both the first end cover 2 and the second end cover 2 nearby the mounting cavity 40 is provided with the support pillar 22, two fans 20 are respectively installed onto the support pillar 22 of the first end cover 2 and the second end cover 2, fans 20 and end covers 2 form a module, fans 20 can be dismounted after removing end covers 2, fans 20 can be installed onto the end covers 2 first, and then the end covers 2 are used to cover the light body 1, which facilitates mounting and dismounting and avoids more complex internal structure inside the light body 1. Specifically, one end of the support pillar 22 is provided with threads, and the fan 20 is fixed onto the support pillar 22 through collaboration between nut 94 and support pillar 22.

Further, as shown in FIG. 14, a filter structure 24 is provided between the end cover 2 and the fan 20 for filtering particles and dust in the air and preventing UV rays being out of the light. Wherein, the filter structure 24 can include the first filter screen and the second filter screen, the first filter screen can be made of foam for adsorbing and filtering small particles, dust and other hazardous substances, the second filter screen is used for filtering large particles and clamping the first filter screen; the support pillar 22 is enclosed by the supporting piece 28 provided between the second filter screen and the end cover 2, preventing failure of the first filter screen arising from excessive extrusion. Of course, only the second filter screen can be provided for filtering large particles, preventing damage to the structure inside the light body 1. Wherein, the middle of the second filter screen is a latticed pore structure, the size of the latticed pore is smaller than 1 mm.

As shown in FIG. 11, 16, 17, 22, in one embodiment, the outer surface of the light body 1 is provided with the third mounting slot 15 for setting the assembling part 9, both sides of the light body 1 are provided with two assembling parts 9 for installing the straight-tube UV light 10 onto the preset position.

Specifically, the assembling part 9 includes the mounting plate body 95 and two bosses 91 provided symmetrically on both ends of the mounting plate body 95 respectively, and two bosses 91 can be a hollow structure that is supported and reinforced by rib slabs on both sides; the first fixing hole 88 is provided on the mounting plate body 95 between two bosses 91 for fixing the assembling part 9 onto the straight-tube UV light 10; the upper surface of at least one boss 91 is provided with a hanging hole 92 for collaborating with external hanging parts; the second fixing hole 89 is provided on the mounting plate body 95 inside two bosses 91, the second fixing hole 89 is opposite to the hanging hole 92 and can also be used to fix the assembling part 9 onto the light. When connecting and fixing the assembling part 9 and the light body 1, bolt 93 goes through the first fixing hole 88, collaborates with the nut 94 to fix the bolt 93, the cap of the bolt 93 is provided inside the third mounting slot as the sliding part 57, assembling part 9 slides onto the preset position, nut 94 is fastened, the assembling part 9 is fixed above the third mounting slot 15, facilitating adjustment of the position of the assembling part 9. In this embodiment, when the straight-tube UV light 10 is placed horizontally, the boss 91 can be used as a supporting leg; when the straight-tube UV light 10 is mounted onto the wall, detachable connection can be realized via the hanging hole 92.

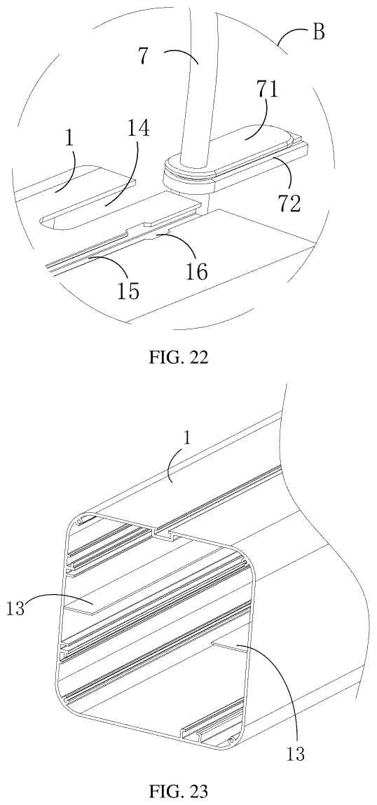

Further, as shown in FIG. 22, one end or two ends of the third mounting slot 15 is/are provided with the avoiding hole 16, when the end cover 2 is not open, the bolt for fixing the assembling part 9 or other fasteners can be directly installed into the third mounting slot 15 via the avoiding hole 16, by which the assembling part 9 can be mounted without dismounting the end cover 2, enabling fast and convenient installation.

In one embodiment, as shown in FIG. 16, functional subassemblies also comprise the reflective plate 55 that is provided around the first light source subassembly 23. Reflective plate 55 is used for reflecting UV rays. After times of reflection, the utilization efficiency of UV rays can be improved.

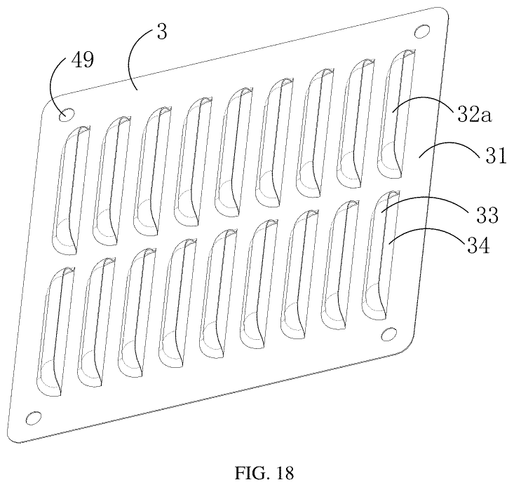

As shown in FIG. 18-20, in one embodiment, the straight-tube UV light 10 also comprises the baffle 3 provided inside the mounting cavity 40, the baffle 3 is provided between the first light source subassembly 23 and one end cover and is vertical to the length of the light body 1; the board 31 of the baffle 3 is provided with a ventilation structure that is provided with the ventilation slot 34 for ventilation, the ventilation slot 34 can prevent UV rays vertical to the plane of the board 31 from being emitted onto the other side of the plane 31, preventing damage to human body caused by UV rays out of the light body 1.

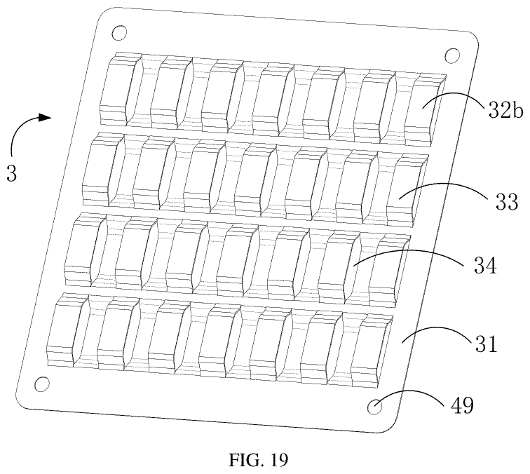

Specifically, the ventilation structure of the baffle 3 comprises multiple convex parts 33 and multiple ventilation slots 34, the ventilation slots 34 are holes for connecting both sides of the board 31, convex parts 33 are a prominent structure on the board 31 for shielding UV rays, the projection of the convex parts 33 on the plane of the board 31 covers the projection of the ventilation slot 34 on the plane of the board 31; according to the distribution of multiple convex parts 33 and multiple ventilation slots 34, the ventilation structure can be a unilateral ventilation structure 32a or a bilateral ventilation structure 32b; as shown in FIG. 18, this is the unilateral ventilation structure 32a, multiple convex parts 33 and multiple ventilation slots 34 are provided on one side of the board 31 of the baffle 3; as shown in FIG. 19, this is the bilateral ventilation structure 32b, multiple convex parts 33 and multiple ventilation slots 34 are respectively provided on one side of the board 31 of the baffle 3; in this embodiment, ventilation is realized with the ventilation slot 34, the opening of the ventilation slot 34 is vertical to the board 31, preventing damage to human body caused by UV rays emitted by the first light source subassembly 23 via the opening of the ventilation slots 34.

It needs to be noted that for convex parts 33 and ventilation slots 34 in the unilateral ventilation structure 32a and the bilateral ventilation structure 32b, it is not required that their shape and connection relationship are completely the same, and the shape and connection relationship of their convex parts 33 and ventilation slots 34 may be different.

In one embodiment, baffle 3 is fixed onto the support pillar 22 of the end cover 2. Sheet parts are hard to be fixed inside the light body 1, and it is hard to operate if it is deep, so the baffle 3 can be fixed onto the end cover 2 before installing the end cover 2 in this embodiment, and then end cover 2 is fixed onto the light body 1, facilitating mounting and dismounting.

In one embodiment, the above-mentioned ventilation structure of baffle 3 can be applied to end cover 2, at least one air hole of the first air hole 21 and the second air hole 21 should be provided as the unilateral ventilation structure 32a, or at least one air hole of the first air hole 21 and the second air hole 21 should be provided as the bilateral ventilation structure 32b, further preventing damage to human body caused by UV rays from the light body 1.

As shown in FIG. 16 and FIG. 20 and FIG. 21, in one embodiment, functional subassemblies also include the support 5 provided inside the mounting cavity 40, and the first light source subassembly 23 and the control power supply 30 are respectively fixed onto the support 5. wherein, the inner surface of the light body 1 is provided with a chute structure 12, the support 5 is provided with a sliding part 57, the sliding part 57 and the chute structure 12 collaborate with each other, enabling that the support 5 can move to the assembly position along the chute structure 12 and then can be fixed into the light body 1 in a dismountable way via the locating device 6. When installing and replacing the first light source subassembly 23 or the control power supply 30, the support 5 can be pulled out of or pushed into the mounting cavity along the chute to improve the convenience of installation and maintenance. Specifically, the cross section of the chute of the chute structure 12 can be in the “T” shape or the “L” shape; the locating device 6 can be multiple pressing strips 61 provided inside the chute, after the support 5 slides to the preset position, one end of the pressing strips 61 is against the sliding part 57, the other end is against the end cover 2 to fix the connecting structure 44, or bolts 93 or other fastening parts are added between the light body 1 and the connecting structure 44 to fix the support 5.

Further, one end of the support 5 is provided with a handle 56 on which there is a hollow groove for firm holding in service, the handle 56 drives the support 5 to be pushed into or pulled out of the light body 1 along the chute structure 12, facilitating movement of the support 5 and winding of lead wire inside the light.

Specifically, the support 5 includes the base plate 58 and the supporting plate 59, the sliding part 57 is provided on one side of the base plate 58, the supporting plate 59 is provided on the other side of the base plate 58; the first light source subassembly 23 comprises the first light source 231 provided on the supporting plate 59 that supports the first light source 231 above the base plate 58 in a suspension way; the control power supply 30 is provided on the base plate 58 and is between the base plate 58 and the first light source 231.

More specifically, the handle 56 is provided with the mounting base 232 that connects to the control power supply 30 in an electric way, and connects to the electric connection end of the first light source 231 and fixes it onto the mounting base 232, supplies power to the first light source 231 via the mounting base 232 and fixes one end of the first light source 231, the other end of the base plate 58 is provided with the supporting plate 59, the upper side of the supporting plate 59 is provided with a protective lantern ring 60 in a nesting way, the other end of the first light source 231 goes through the protective lantern ring, and the supporting plate 59 and the protective lantern ring 60 fix the other end of the first light source 231.

Of course, both ends of the base plate 58 can be provided with the supporting plate 59, the first light source 231 goes through two supporting plates 59, so that the first light source 231 can be fixed firmly.

Wherein, the first light source 231 can be a UV light tube or UV LED module.

As shown in FIG. 21 and FIG. 22, in one embodiment, one end of the light body 1 is provided with the fixing opening 14 that can be a “U” shaped gap, the connecting part of the power cable 7 and the light body 1 of the straight-tube UV light 10 can be provided with a fixing block 71, the lateral side of the fixing block 71 corresponding to the fixing opening 14 is provided with the second mounting slot 72, the fixing block 71 slides inside the fixing opening 14 via the second mounting slot 72, when the fixing block 71 slides to the extreme position inside the fixing opening 14, the fixing block 71 completely covers the fixing opening 14, and the end cover 2 is against the fixing block 71 to fix the power cable 7.

In one embodiment, the chute structure 12 can be realized in the way where the chute is provided on the light body 1 and the slide rail is provided on the support 5; or a slide rail can be provided on the light body 1, and a chute is provided on the support 5; or other locating structures are provided on the inner surface of the light body 1, the corresponding device on the support 5 can push the support 5 to other structures at the preset position on the light body 1, as shown in FIG. 23, the inner surface of the light body 1 is provided with a supporting face 13, the support 5 can slide on the supporting face 13, and the support 5 is fixed onto the light body 1 after it slides to the preset position. All these schemes can make mounting and dismounting of the support 5 more convenient and faster.

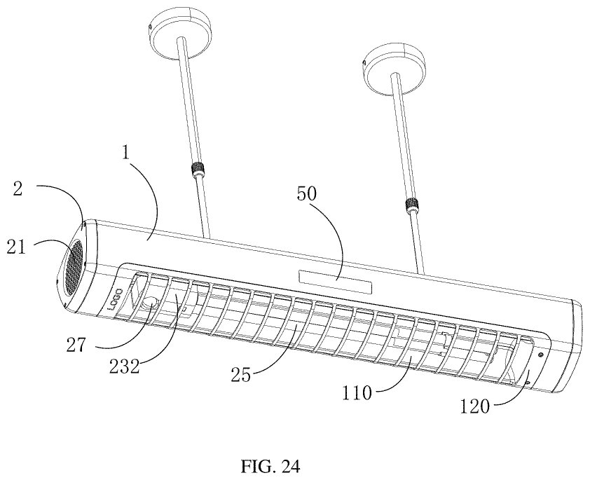

FIG. 24-FIG. 28 show the straight-tube UV light 10 that combines the ventilation-type scheme and the direct irradiation scheme.

As shown in FIG. 24-FIG. 26, in one embodiment, the lower part of the light body 1 is provided with the second opening 19, the straight-tube UV light 10 also comprises the second cover plate 110 that covers the second opening 19 for sealing, the top of the cover plate faces the inside of the mounting cavity 40, the bottom of the cover plate faces the downward (outside), the second cover plate 110 and the light body 1 are connected in a dismountable way, so that the second cover plate 110 can cover or open the second opening 19. By opening the second cover plate 110, the structure inside the light body 1 or on the second cover plate 110 can be dismounted, making repair, maintenance or replacement of parts inside the light body 1 or on the cover plate more convenient.

Wherein, the second cover plate 110 can connect to the light body 1 via the hinge or in other connection way, the first light source subassembly 23 and the control power supply 30 are fixed onto the top of the second cover plate 110, by opening the second cover plate 110, the first light source subassembly 23 and the control power supply 30 can be replaced and installed, enabling convenient operation. Of course, the second cover plate 110 can be opened to expose the internal structure of the light body 1, so the first light source subassembly 23 and the control power supply 30 can be fixed onto the inner surface of the light body 1. In other embodiments, the second opening 19 can also be provided on different surfaces of the light body, the second opening 19 is provided with other functional components and parts, such as light source, fan, protective net shield, control module and optical subassembly to realize corresponding functions.

Further, the side of the first end cover 2 nearby the mounting cavity 40 is provided with the fan 20, or/and, the side of the second end cover 2 nearby the mounting cavity 40 is provided with the fan 20 to improve the air supply efficiency and improve the sterilization and disinfection efficiency of the straight-tube UV light 10.

Further, the straight-tube UV light 10 comprises the second light source module 25 provided at the bottom of the second cover plate 110, and the second light source module 25 directly emits UV rays or visible light to the external environment; when the second light source module 25 emits UV rays, the straight-tube UV light can enable the ventilation type and the direct irradiation sterilization and disinfection, significantly improving the sterilization and disinfection efficiency of the light, the ventilation mode can be applied when there are people on the sterilization site, and the direct irradiation mode can be applied when there is no people on the sterilization site; when the second light source module 25 emits visible light, the straight-tube UV light can enable the sterilization and disinfection and lighting and emits visible light below provide lumination, realizing two functions of one light. In such a case, the second cover plate 110 can serve as a reflective housing whose middle is provided with a reflective concave reflective slot, the second light source module 25 is provided inside the concave reflective slot which reflects via the concave reflective slot the light emitted onto the second cover plate 110 to improve the illuminating rate.

Further, the straight-tube UV light 10 also comprises the protective net shield 120 that covers the second cover plate 110 and the second light source module 25. The protective net shield 120 prevents foreign objects or human bodies from touching the second light source module 25, so that foreign objects will not damage the second light source module 25 or the second light source module 25 brings damage to human bodies.

In one embodiment, the second light source module 25 comprises the mounting base 232 and the second light source 26, the mounting base 232 is provided with a sensing device 27 which comprises a signal receiving element that is used for receiving the external control signal (e.g. control signal of the remote control), the control power supply 30 controls work of the straight-tube UV light 10 according to the control signal, by which the working condition, working mode of the straight-tube UV light 10 can be controlled or its working parameters can be adjusted from a distance, avoiding exposure of human body to the UV light source.

Further, the infrared sensor or microwave sensor can be provided inside the sensing device 27, when human body approaches the working light, the sensor will automatically turn off the second light source 26 to ensure safety of the user.

In one embodiment, the straight-tube UV light 10 also comprises a display 50 electrically connecting to the control power supply 30, the display 50 can display the working condition of the straight-tube UV light 10, so that the working condition of the light can be monitored and displayed. For example,

(1) After the sterilization time of the remote control is set, the display 50 displays the sterilization time of the light;

(2) After the fan gear is set with the remote control, the gear FIG. of the fan is displayed;

(3) When the air filter screen reaches the preset service life, it will be prompted that the air filter screen should be replaced timely;

(4) When the accumulative service time of the light source reaches the preset service life, it is prompted that the light source should be replaced timely.

In one embodiment, the top of the light body 1 is also provided with the third opening 140, the straight-tube UV light 10 also comprises the third cover plate 150 that covers the third opening 140, and the third cover plate 150 is provided with the third air hole 21; the first air hole 21 and the second air hole 21 are respectively provided with a fan 20, two fans 20 blow air to the outside of the light body 1, under the action of two fans 20, the first air hole 21 and the second air hole 21 serve as the air outlet, the third air hole 21 serves as the air inlet, so that air can be flow from the top to the bottom, promoting circulation of the air around the light, air flows into the light body 1 via the middle third air hole 21, passes through the first light source 231 for sterilization and disinfection, and then is exhausted to the outside of the light body 1 via the first air hole 21 and the second air hole 21, improving the sterilization and disinfection efficiency of the straight-tube UV light 10.

Further, the side of the third cover plate 150 nearby the accommodating cavity is also provided with a filter structure 24 that covers the third air hole 21 and filters air that flows into the light body 1, improving the air purification effect of the light and facilitating replacement of the filter structure 24.

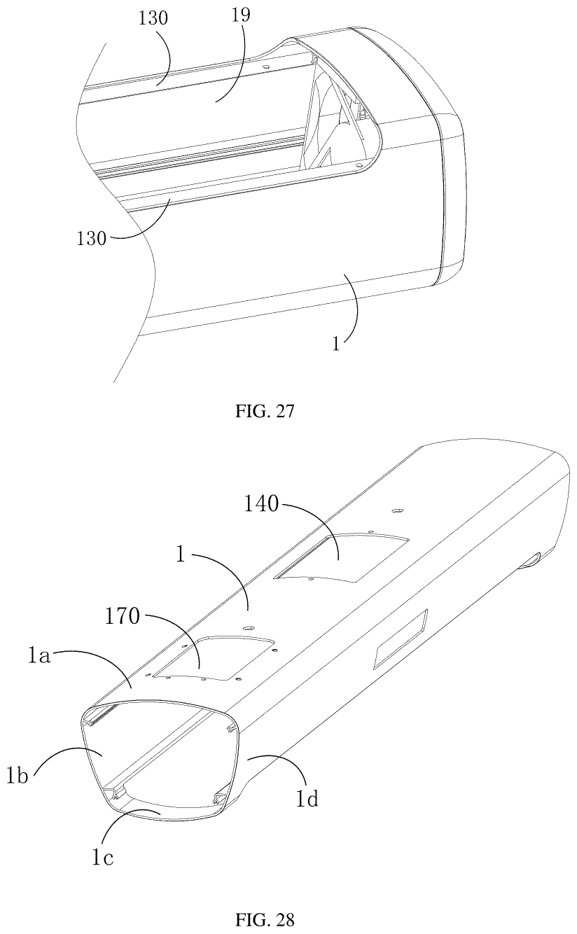

As shown in FIG. 27, in one embodiment, the inner wall of the light body 1 corresponding to the second opening 19 is provided with two convex ribs 130 that are respectively nearby the two opposite surfaces of the second opening 19, when the second cover plate 110 covers the second opening 19, two convex ribs 130 shield the mounting joint of the second cover plate 110 and the second opening 19, preventing leakage of the UV ray and improving the supporting strength of the second cover plate 110 during installation.

As shown in FIG. 28, in one embodiment, the light body 1 is a straight-tube structure formed through enclosing of the first arc surface 1a, the second arc surface 1b, the third arc surface 1c and the fourth arc surface 1d, since four arc surfaces can reflect UV rays, the utilization efficiency of the UV light source inside the light body 1 form by four arc surfaces can be improved.

Wherein, the first arc surface 1a is provided with the third opening 140 and the wiring hole 170 for connecting lead wire, the power cable 7 goes through the wiring hole 170 and connects to the control power supply 30 to supply power for the light; the mounting opening of the display 50 is provided on the fourth arc surface 1d; the second opening 19 is provided on the third arc surface 1c.

The above description only presents the preferred embodiments of the present invention, and it is not for this reason that the patent scope of the invention is limited. Any equivalent structural transformation made by using the description of the invention and the drawings, or direct/indirect application in other related technical fields under the inventive concept of the invention, is included in the patent protection scope of the invention.

What is claimed is:

1. A straight-tube UV light, comprising:
a light body of a hollow tubular structure provided in a straight line, defined as that a length of the light body that extends from left end to right end, a height that extends up from a bottom and a width that extends from a front to back;
end covers, including a first end cover and a second end cover provided on the left end and the right end of the light body, the first end cover, the second end cover and the light body enclosing to form a mounting cavity, the first end cover and the second end cover respectively provided with a first air hole and a second air hole connecting to the mounting cavity; and
functional subassemblies, including at least a first light source subassembly and the control power supply, wholly provided inside the mounting cavity, the control power supply used for controlling the first light source subassembly to emit UV rays for sterilization and disinfection.

2. The straight-tube UV light as claimed in claim 1, wherein the front side of the light body is provided with a first opening, the functional subassemblies also comprise a grating subassembly provided in the mounting cavity and between the first light source subassembly and the first opening; and
the grating subassembly comprises multiple baffles provided at intervals, adjacent the baffles form a light emitting slot along which the first light source subassembly emit UV rays out of the light body.

3. The straight-tube UV light of claim 2, wherein the functional subassemblies also comprise a support provided inside the mounting cavity, the first light source subassembly and the control power supply are fixed onto the support;
the support comprises a reflective part and a connecting part, the reflective part is an arc structure, a concave surface of the reflective part is opposite to the first opening, the first light source subassembly is provided in the concave surface of the reflective part; and
an inner surface of the light body is provided with a chute structure, the connecting part is provided with a sliding part, the sliding part and the chute structure collaborate with each other to make the support move to a preset assembly position along the chute structure, and the support is fixed into the light body in a dismountable way via the locating device.

4. The straight-tube UV light of claim 3, wherein the concave surface of the reflective part is defined as a front, and an opposite surface of the concave surface is a back; the connecting part comprises two supporting arms provided at intervals, the two supporting arms are provided on the back of the reflective part, forming the first mounting slot, and the control power supply is fixed inside the first mounting slot; and
the end of each of the supporting arm far from the reflective part is provided with a sliding part.

5. The straight-tube UV light of claim 3, wherein the straight-tube UV light also comprises a handle provided on one end of the support, the handle drives the support to be pushed into or pulled out of the light body along the chute structure.

6. The straight-tube UV light of claim 5, wherein the first light source subassembly comprises the first light source and a mounting base, the mounting base is configured for fixing the first light source and supplying power to the first light source; and
the mounting base is fixed onto the handle, and the first light source and the reflective part are provided at intervals.

7. The straight-tube UV light of claim 2, wherein the back of the light body is provided with another opening, the first opening on the back of the light body is also provided with another grating subassembly that enables the UV rays to be emitted horizontally from the back of the light body.

8. The straight-tube UV light of claim 2, wherein a board of the baffles is provided in a curved shape, an upper surface of the baffles are provided with multiple upper convexes, a lower surface of the baffles is provided with multiple lower convexes, and at least parts of the upper convexes and the lower convexes are connected in sequence to form a continuous waveform structure.

9. The straight-tube UV light of claim 8, wherein the baffles comprise two continuous waveform structures, a middle part of the board is provided horizontally, two the continuous waveform structures are connected with horizontal boards, a middle part of the horizontal boards is provided with mounting holes, and the mounting holes are used for fixing baffles during installation.

10. The straight-tube UV light of claim 2, wherein the grating subassembly comprises at least one connecting structure, each of the at least one connecting structure comprises a central column and multiple connecting columns, multiple baffles and multiple connecting columns enclose the central column, and the connecting columns are provided between two adjacent the baffles; and
a bottom of the central column is provided with a sliding part, the inner surface of the light body is provided with a chute structure, the sliding part and the chute structure collaborate with each other to make the grating subassembly move along the chute structure to preset assembly position, and the grating subassembly is fixed onto the light body in a dismountable way via the locating device.

11. The straight-tube UV light of claim 1, wherein the side of the first end cover or/and the second end cover nearby the mounting cavity are provided with a support pillar; and
the functional subassemblies comprise a fan fixed onto an end cover via a support pillar, wherein the fan is corresponding to the first air hole or/and the second air hole.

12. The straight-tube UV light of claim 1, wherein the tubular UV light further comprises a sensing module electrically connected to the control power supply, and the sensing module is provided below the front surface of the light body; wherein the sensing module is an infrared sensing module, and when human body reaches the sensing height of the sensing module, the sensing module automatically turns off the first light source;
or/and, the functional subassemblies further comprise a support provided inside the mounting cavity, the support comprises a reflective part and a reflective plate, the reflective part is an arc structure, the reflective plate is against the concave surface of the reflective part;
or/and, the straight-tube UV light further comprises a power cable, the connecting part of the power cable and the light body is provided with a fixing block, the fixing block is provided with the second mounting slot; the end surface of the light body is provided with a fixing opening, the fixing block slides within the fixing opening via the second mounting slot; wherein when the fixing block slides to the limiting position inside the fixing opening, the fixing block covers the fixing opening, and the end cover is against the fixing block to fix the power cable;
or/and, the outer surface of the light body is provided with the third mounting slot, the third mounting slot is used for setting assembling parts via which the straight-tube UV light is installed onto the preset position.

13. The straight-tube UV light of claim 12, wherein one end of the third mounting slot is also provided with an avoiding hole, wherein when the end cover is not opened, the assembling parts are installed into the third mounting slot via the avoiding hole.

14. The straight-tube UV light of claim 12, wherein the sensing module comprises a sensing element, a fixing base, a lens and a circuit board, the sensing element is provided onto the circuit board, the fixing base is provided with the first go-through slot, the circuit board is fixed onto the fixing base, the sensing element is placed in a first go-through slot, the lens covers another end fixed into the first go-through slot;
the lens is provided in a square shape, a side of the lens corresponding to the sensing element is provided with a square slot, the bottom surface of the square slot is provided with several circles of convex patterns; and
the inner surface of the light body is provided with the first arc surface, the fixing base comprises a convex block where there is a second arc surface, the second arc surface is against the first arc surface, the lower part of the front surface of the light body is provided with the second pass-through slot, the lens is inserted into the second pass-through slot, by which the sensing module is fixed onto the light body.

15. A straight-tube UV light comprising:
a light body of a hollow tubular structure provided in a straight line, defined as a length of the light body extending from left to right, a height extends from up to a bottom, and a width extends from a front to a back;
an end cover, including a first end cover and a second end cover provided on the left and the right of the light body, the first end cover, the second end cover and the light body enclosing to form a mounting cavity; and
functional subassemblies, including at least the first light source subassembly and a control power supply, wholly provided inside the mounting cavity, the control power supply configured to control the first light source subassembly to emit UV rays for sterilization and disinfection.
wherein the front side of the light body is provided with a first opening, the functional subassemblies further comprise a grating subassembly provided inside the mounting cavity and between the first light source subassembly and the first opening; the grating subassembly comprises multiple baffles provided at intervals, and adjacent baffles form a light emitting slot along which the first light source subassembly emit UV rays out of the light body.
